(19) United States
(12) Patent Application Publication
(10) Pub. No.: US 2016/0128548 A1
(43) Pub. Date: May 12, 2016

(54) LARYNGOSCOPE

(71) Applicant: Hsien-Yung LAI, Hualien County (TW)

(72) Inventor: Hsien-Yung LAI, Hualien County (TW)

(21) Appl. No.: 14/937,900

(22) Filed: Nov. 11, 2015

(30) Foreign Application Priority Data
Nov. 12, 2014 (TW) 103139266

(51) Int. Cl. A61B 1/00; A61B 1/04; A61B 1/267

(52) U.S. Cl. CPC A61B 1/00101; A61B 1/267; A61B 1/00105; A61B 1/04; A61B 1/00117; A61B 1/00045

(57) ABSTRACT

A laryngoscope of some embodiments includes a handle, a first blade, and a second blade. The first blade is capable of being detachably coupled with the handle. The second blade is capable of being detachably coupled with the handle, in which the first blade and the second blade have different forms.

### CROSS-REFERENCE TO RELATED APPLICATION

This application claims the priority benefit of Taiwanese application serial no. 103139266, filed Nov. 12, 2014, the full disclosure of which is incorporated herein by reference.

### BACKGROUND

1. Technical Field

The present disclosure relates to laryngoscopes.

2. Description of Related Art

Endotracheal intubation is inserting an endotracheal tube from a mouth or a nasal cavity, through the throat and the glottis, and into the trachea, whereby an artificial open airway is established. Common causes of intubation include respiratory failure and respiratory track having no self-protection. In early times, endotracheal intubation is performed by fingers groping. Latter, direct laryngoscope began to be widely used. Recently, videos through optical fibers were added into the direct laryngoscopes to become video laryngoscopes.

However, no matter which kind of laryngoscope, all cannot exchange the blades thereof to meet the various requirements of different patients. For example, the blades with greater angles help difficult intubation cases more but the technical barrier of the intubation is relatively higher. Therefore, for general cases, if a user wants to use a laryngoscope with a smaller angle, the only choice is to use a laryngoscope of another brand or model. This problem is especially serious for the video laryngoscopes, since the video laryngoscopes are much more expensive. Therefore, if a hospital often needs to purchase a special laryngoscope of a certain brand or model for a special case, that will be a great spending.

### SUMMARY

According to some embodiments of the present disclosure, laryngoscope includes a handle, a first blade and a second blade. The first blade is capable of being detachably coupled with the handle. The second blade is capable of being detachably coupled with the handle, in which the first blade and the second blade have different forms.

### BRIEF DESCRIPTION OF THE DRAWINGS

FIG. 1 is a perspective view of a laryngoscope according to some embodiments of the present disclosure.

FIG. 2 is an exploded view of the laryngoscope in FIG. 1.

FIG. 3 is perspective view of modifying the laryngoscope in FIG. 1 by a second blade.

FIG. 4 is a perspective view of modifying the laryngoscope in FIG. 1 by a third blade.

FIG. 5 is a perspective view of modifying the laryngoscope in FIG. 1 by a fourth blade.

FIG. 6 is a functional block diagram of the laryngoscope in FIG. 1.

### DETAILED DESCRIPTION

In the following detailed description, for purposes of explanation, numerous specific details are set forth in order to provide a thorough understanding of the disclosed embodiments. It, will be apparent, however, that one or more embodiments may be practiced without these specific details. In other instances, well-known structures and devices are schematically shown in order to simplify the drawing.

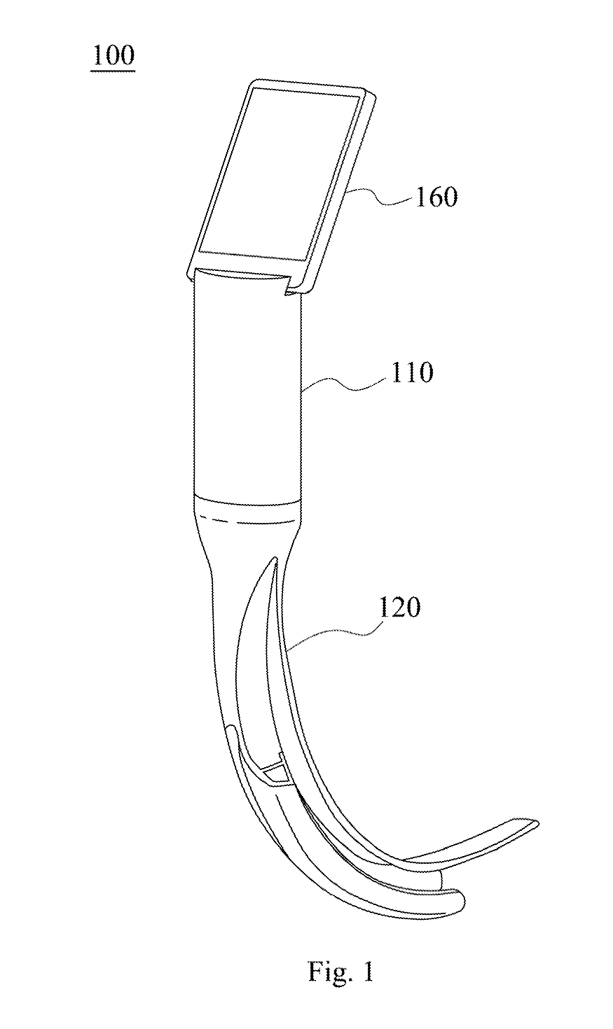

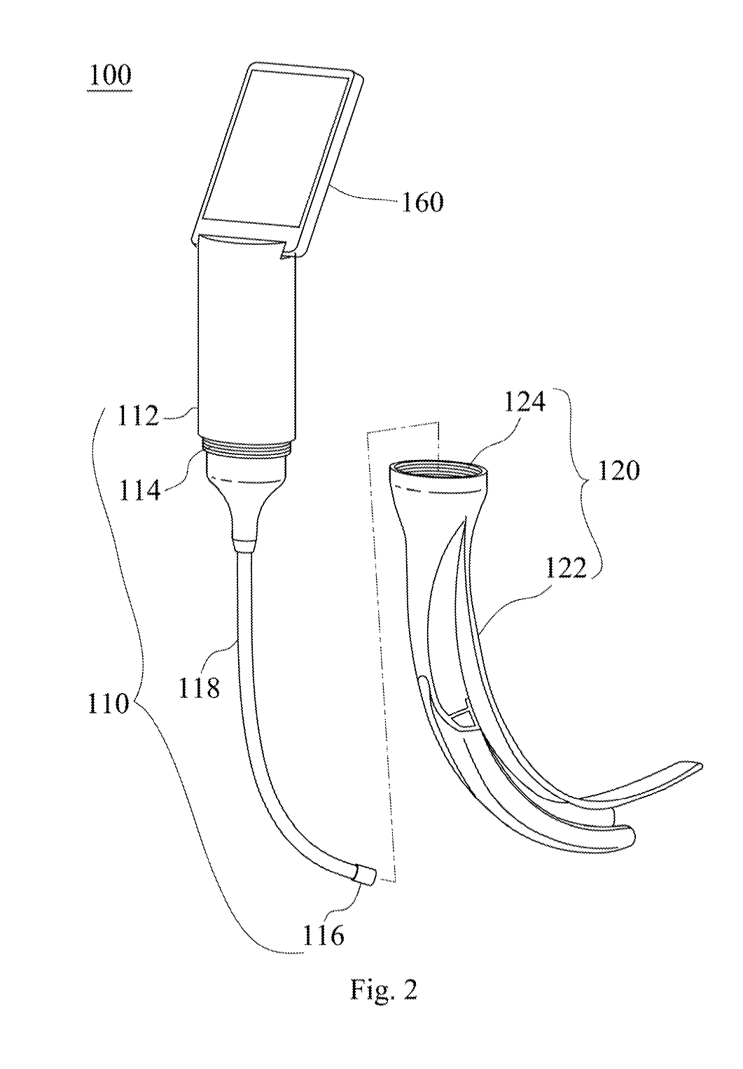

FIG. 1 is a perspective view of a laryngoscope 100 according to some embodiments of the present disclosure. FIG. 2 is an exploded view of the laryngoscope 100 in FIG. 1. As shown in FIG. 1 and FIG. 2, the laryngoscope 100 includes a handle 110 and a first blade 120. The first blade 120 is capable of being detachably coupled with the handle 110.

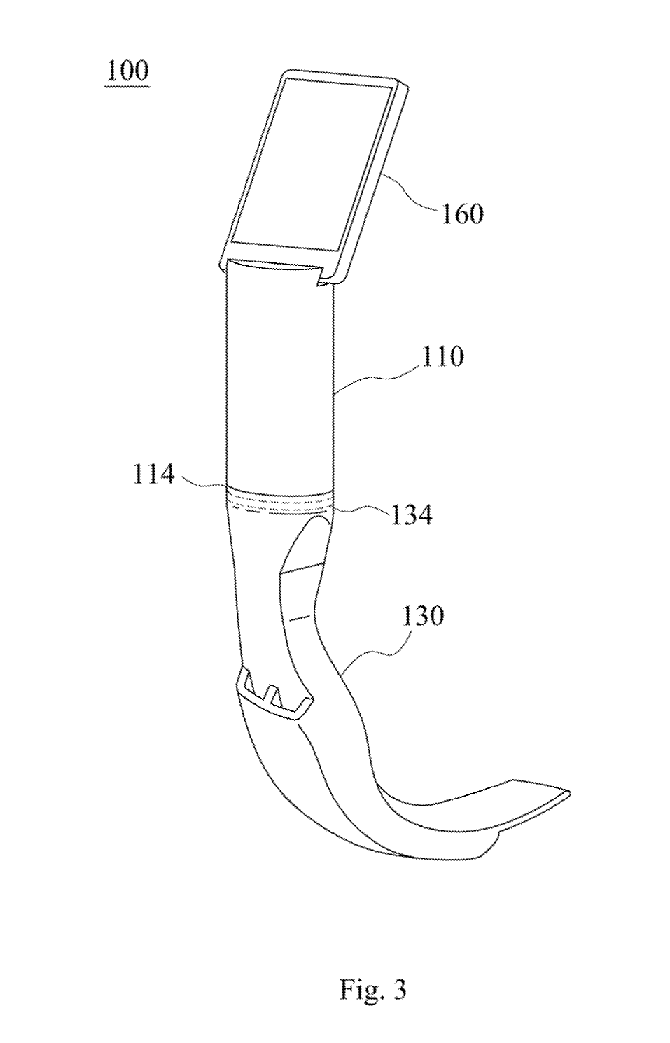

In some embodiments, the first blade 120 has a smaller angle, such as about 50°, and thus has a lower technical barrier and is suitable for general cases. When a more difficult case needs to be intubated, the first blade 110 is removed first and a second blade is installed instead. As shown in FIG. 3, the second blade 130 is also capable of being detachably coupled with the handle 110, but the second blade 130 has a different form from the first blade 120. More specifically, the angle of the second blade 130 is greater than the angle of the first angle 120. The angle of the second blade 130 is about 65°, for example. Therefore, the second blade 130 can provide more help to the more difficult case. Moreover, the second blade 130 also has a different shape from the first blade 120.

In this embodiment, the meaning of “detachably coupling” is that “when a user detaches two elements coupled together, at least one of the two elements is not damaged.” For example, since the first blade 120 is detachably coupled to the handle 110, at least the handle 110 is not damaged when the first blade 120 is detached from the handle 110. Therefore, the second blade 130 can still be installed to the handle 110 for the next operation.

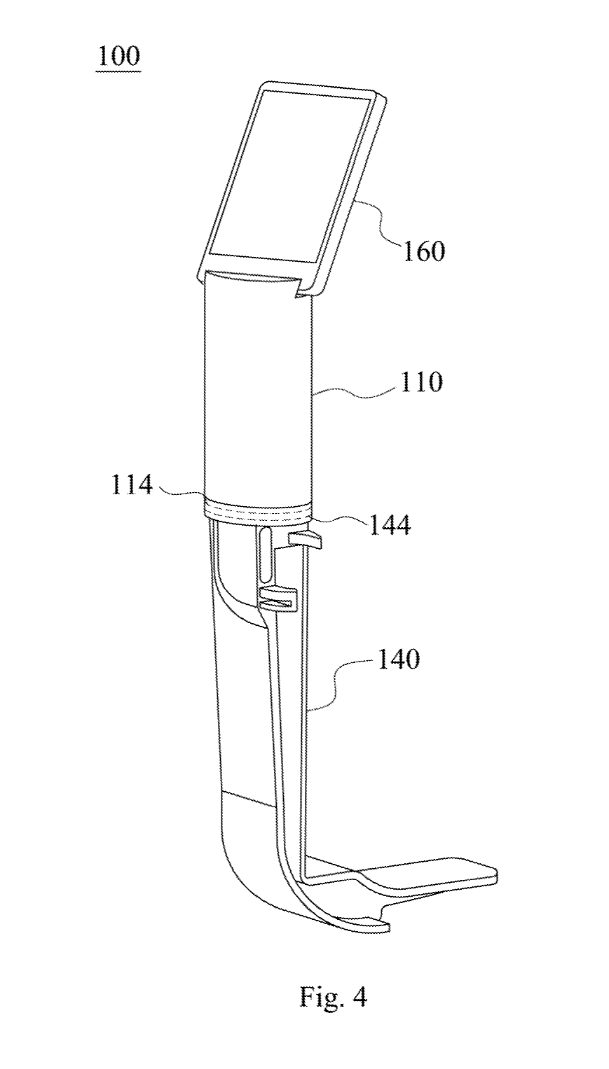

In addition, a third blade can also be installed to the handle 110. As shown in FIG. 4, the third blade 140 is also capable of being detachably coupled with the handle 110, but the third blade 140 has a different shape from the first blade 120 and the second blade 130. Moreover, the angle of the third blade 140 is smaller than the angle of the first blade 120. Therefore, the third blade 140 has a lower technical barrier than the first blade 20 and thus provides less help to the difficult intubation case.

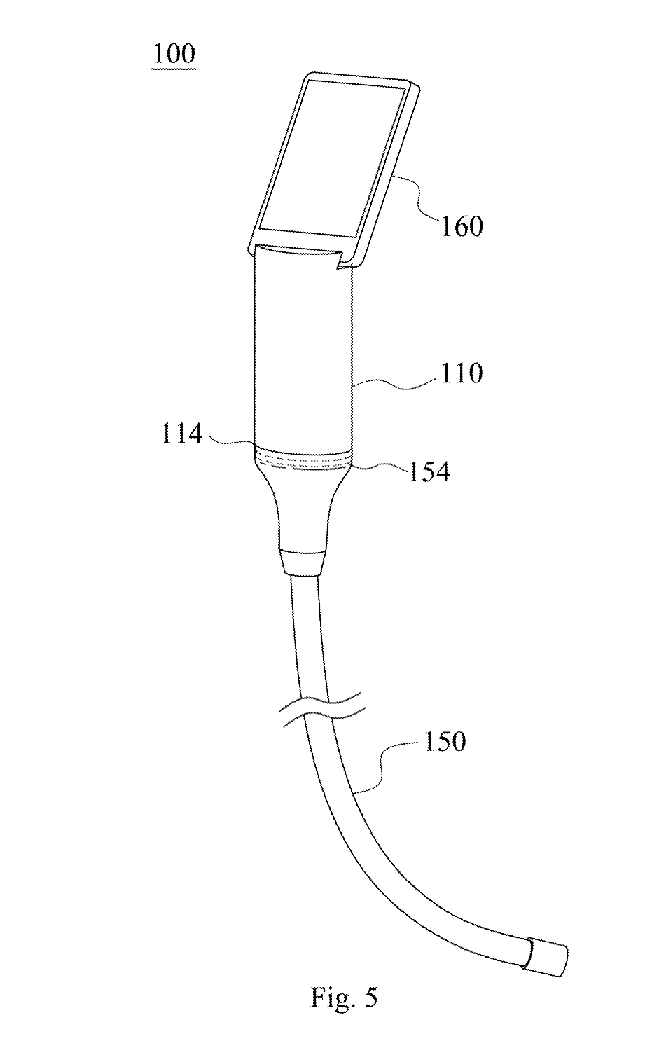

Furthermore, a fourth blade in a form of intubating stylet can also be installed to the handle 110. As shown in FIG. 5, the forth blade 150 is also capable of being detachably coupled, with the handle 110, but the fourth blade 150 has a different shape from the first blade 120 the second blade 130, and the third blade 140, and certainly has an even different size. The forth blade 150 in the form of intubating stylet is suitable to be used in some special cases, such as cases of snaggleteeth, wobbling teeth, or small mouth opening.

In practice, at least one or all of the first blade 120, the second blade 130, the third blade 140, and the forth blade 150 are disposable to avoid the inconvenience of resterilizing and the derived risk of infection. In this disclosure, the meaning of “disposable” is “discarding after one or several operations without undue resterilization.” Generally speaking, a disposable blade will be discarded and replaced by a new one after one or several operations. Hence, a cheaper material, such as plastic, will be chosen.

In some embodiments, the first blade 120 has a first coupling feature 124, the second blade 130 has a second coupling feature 134, the third blade 140 has a third coupling feature 144, and the fourth blade 150 has a fourth coupling feature 154. The handle 110 has a handle coupling feature 114. All of the first coupling feature 124, the second coupling feature 134, the third coupling feature 144, and the fourth coupling feature 154 can be coupled with the handle coupling feature 114.

Since the first coupling feature 124, the second coupling feature 134, the third coupling feature 144 and the fourth coupling feature 154 can be coupled with the handle coupling feature 114, the first coupling feature 124, the second coupling feature 134, the third coupling feature 144 and the fourth coupling feature 154 are substantially the same, whereby the first blade 120, the second blade 130, the third blade 140, and the fourth blade 150 can be detachably coupled with the handle 110.

The first coupling feature 124, the second coupling feature 134, the third coupling feature 144 and the fourth coupling feature 154 can be coupled with the handle coupling feature 114 without using any tool. Therefore, no additional tool is needed to detach the first blade 120, the second blade 130, the third blade 140 and the fourth blade 150, and the detaching operation can be performed more smoothly. In some embodiments, the first coupling feature 124, the second coupling feature 134, the third coupling feature 144 and the fourth coupling, feature 154 are internal threads of the first blade 120, the second blade 130, the third blade 140, and the fourth blade 150, respectively. The handle coupling feature 114 is an external thread of the handle 110. Of course other tool-free coupling features, such as tightly-matched coupling features, clamp coupling features, spring coupling features, are also applicable. Persons having ordinary skills in the art can flexibly choose the coupling features depending on the actual needs.

The coupling feature 114 can disposed on one side of the handle 110 near the first blade 120, the second blade 130, the third blade 140, or the fourth blade 150. The first coupling feature 124, the second coupling feature 134 the third coupling feature 144 and the fourth coupling feature 154 are respectively disposed on one side of the first blade 120, the second blade 130, the third blade 140, and the fourth blade 150 near the handle 110. Using FIG. 2 as an example, the handle 110 includes a main body 112 and a coupling feature 114, The first blade 120 also includes a main body 122 and a first coupling feature 124. The handle coupling feature 114 is disposed on one side of the main body 112 near the first blade 120, and the first coupling feature 124 is disposed on one side of the main body 122 near the handle 110.

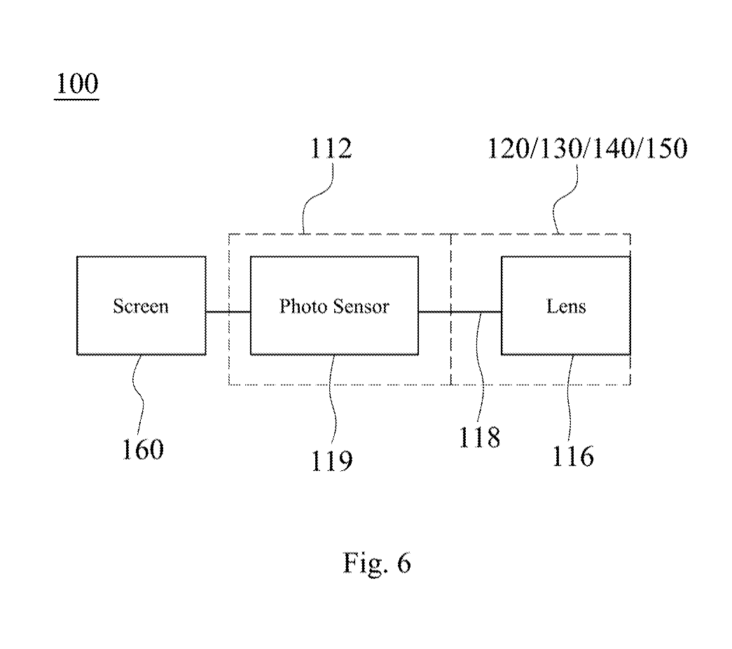

In some embodiments, the laryngoscope 100 is an image laryngoscope. FIG. 6 is a functional block diagram of the laryngoscope 100 in FIG. 1. Referring to FIG. 1 and FIG. 6 simultaneously, the laryngoscope 100 further includes a screen 160. The handle 110 further includes a lens 116, an optical fiber 118, and a photo sensor 119. The optical fiber 118 couples the lens 116 with the photo sensor 119. The photo sensor 119 is used to transform images obtained by the lens 130 to electronic signals. The screen 160 is electrically coupled with the photo sensor 119 to display the images on the screen 160 according to the electronic signals. Therefore, users can observe the intubation status from the screen 160.

In some embodiments, the photo sensor 119 is accommodated in the main body 112. That is, the main body 112 has a cavity, and the photo sensor 140 can be accommodated in the cavity of the main body 112. Furthermore, when the electricity of the laryngoscope 100 is from batteries, such as rechargeable batteries or primary batteries, the batteries can also be installed in the main body 112. Certainly, the electricity of the laryngoscope 100 can also be from mains electricity. When the electricity of the laryngoscope 100 is from mains electricity, a power cable can also be coupled with the main body 112.

In some embodiments, the first blade 120, the second blade 130, the third blade 140, and the fourth blade 150 may have a channel therein. When the first blade 120, the second blade 130, the third blade 140, or the fourth blade 150 is installed on the handle 110, the lens 116 will pass through the channel of the first blade 120, the second blade 130, the third blade 140, or the fourth blade 150 to reach one side of the first blade 120, the second blade 130, the third blade 140, or the fourth blade 150 away from the handle 110. The optical fiber 118 couples the lens 116 with the photo sensor 119 through the channel of the first blade 120, the second blade 130, the third blade 140, or the fourth blade 150.

Practically the photo sensor 119 may be a charge-coupled device (CCD), active-pixel sensor (APS), complementary metal-oxide-semiconductor (CMOS) image sensor, or any combinations thereof.

The screen 160 may be directly disposed on the main body 112. That is, the screen is directly coupled with the main body 112 and no wire exists between the screen 160 and the main body 112. This approach can simplify the entire device. Therefore, only one element of the laryngoscope 100 is needed to be held when operating, no other component is needed to fix the screen 160. In some other embodiments, the screen 160 can also be separated from the main body 112 and is interconnected to the main body 112 by a wire. Persons having ordinary skills in the art can flexibly choose the connecting way between the screen 160 and the main body 112 according to the actual needs.

In some embodiments, the screen 160 is disposed on a terminal of the main body 112 away from the first blade 120, the second blade 130, the third blade 140, or the fourth blade 150. Consequently, when the laryngoscope 100 is used in intubating, the screen 160 would face the user to facilitate the user's observation.

Therefore, the higher-price components of the laryngoscope 100, such as the handle 110 and the screen 160, will be shared by the first blade 120, the second blade 130, the third blade 140, and the fourth blade 150. When a hospital needs to prepare laryngoscopes, only relatively cheap blades are needed to be purchased. The expensive screens, lens, optical fibers, and photo sensors do not need to be purchased repeatedly.

What is claimed is:

1. A laryngoscope, comprising:
a handle;
at least a first blade capable of being detachably coupled with the handle; and
at least a second blade capable of being detachably coupled with the handle, wherein the first blade and the second blade have different forms.

2. The laryngoscope of claim , wherein the first blade and the second blade have different shapes.

3. The laryngoscope of claim 1, wherein the first blade and the second blade have different angles.

4. The laryngoscope of claim 1, wherein the first blade is disposable.

5. The laryngoscope of claim 1, wherein the second blade is disposable.

6. The laryngoscope of claim 1, wherein the first blade has a first coupling feature, the second blade has a second coupling feature, the handle has a handle coupling feature, and both the first coupling feature and the second coupling feature can be coupled with the handle coupling feature.

7. The laryngoscope of claim 6, wherein the first coupling feature and the second coupling feature are substantially the same.

8. The laryngoscope of claim 1, wherein the first blade has a first coupling feature, the second blade has a second coupling feature, the handle has a handle coupling feature, and both the first coupling feature and the second coupling feature can be coupled with the handle coupling feature without using a tool.

9. The laryngoscope of claim , wherein the handle comprising:
a main body;
a photo sensor accommodated in the main body;
a lens; and
an optical fiber coupling the photo sensor with the lens through at least one of the first blade and the second blade to a side thereof away from the handle when said one of the first blade and the second blade is detachably coupled to the handle.

10. The laryngoscope of claim 9, further comprising a screen being electrically coupled with the photo sensor and directly disposed on the main body.
